(19) United States
(12) Patent Application Publication
(10) Pub. No.: US 2025/0213287 A1
(43) Pub. Date: Jul. 3, 2025

(54) GUIDED RE-ENTRY CATHETER SYSTEM FOR CROSSING ARTERIAL CHRONIC OCCLUSIONS

(71) Applicants: Kevin Croce, Dedham, MA (US); Nick Rydberg, Minneapolis, MN (US); Patrick Boldenow, Minneapolis, MN (US)

(72) Inventors: Kevin Croce, Dedham, MA (US); Nick Rydberg, Minneapolis, MN (US); Patrick Boldenow, Minneapolis, MN (US)

(21) Appl. No.: 19/004,081

(22) Filed: Dec. 27, 2024

Related U.S. Application Data
(60) Provisional application No. 63/615,120, filed on Dec. 27, 2023.

(51) Int. Cl. A61B 18/08; A61B 18/00

(52) U.S. Cl. CPC A61B 18/08; A61B 2018/00077; A61B 2018/00208; A61B 2018/00345; A61B 2018/00601; A61B 2018/00702; A61B 2018/00761

(57) ABSTRACT

The present disclosure is a magnetically guided re-entry catheter system and method configured to direct subintimal crossing and re-entry into the artery true lumen of a guidewire during treatment of arterial chronic total occlusions. A magnet is affixed to a catheter, and the catheter is directed through the artery containing the chronic total occlusion via a guidewire. A second, ferromagnetic guidewire is directed through the artery to the opposite side of the chronic total occlusion, before crossing out of the true lumen into the subintimal. The magnetic attraction between the second guidewire and the magnet directs re-entry of the second guidewire into the true lumen once the second guidewire has passed the chronic total occlusion.

### RELATED APPLICATION

The present application claims the benefit of U.S. Provisional Patent Application No. 63/615,120, entitled “GUIDED RE-ENTRY CATHETER SYSTEM FOR CROSSING ARTERIAL CHRONIC OCCLUSIONS”, filed Dec. 27, 2023, which is herein incorporated by reference in its entirety.

### TECHNICAL FIELD

The present invention generally relates to a magnetically guided re-entry catheter system, more specifically an energized ferromagnetic guidewire re-entry catheter system.

### BACKGROUND

Atherosclerosis is the leading cause of vascular disease, which accounts for 38% of all deaths in North America. Coronary artery disease is the leading cause of death from atherosclerosis, with over 500,000 deaths per year in the US alone. Peripheral artery disease affects more than 200 million persons worldwide, including at least 8.5 million persons in the United States. Major complications of atherosclerosis include angina, heart attack, stroke, claudication, limb ischemia, amputation, and death. A common symptom of coronary artery and peripheral artery disease are chronic total occlusions.

Coronary and peripheral artery chronic total occlusions completely obstruct flow within an artery, resulting in significant morbidity and mortality. Coronary artery chronic total occlusions occur in 18% to 52% of patients with coronary artery disease. Peripheral artery intervention in patients with chronic total occlusions open blocked coronary arteries and provide symptom relief, improved left ventricular function, reduced risk of arrhythmias, and better tolerance of acute coronary syndromes. Failed percutaneous coronary and peripheral artery intervention (PCI) are associated with increased angina, depression, and death compared with successful chronic total occlusion recanalization. Peripheral artery chronic total occlusions are also common and occur in >50% of patients undergoing peripheral artery disease treatment.

Chronic total occlusions are a major challenge in coronary and peripheral artery treatment. Despite advanced algorithms for chronic total occlusion percutaneous interventions, success rates are less than 60%. The US PCI database demonstrates that coronary chronic total occlusion PCIs use more contrast volume and fluoroscopy time, have lower success (59% vs. 96%), and have higher adverse cardiac events vs. non-chronic total occlusion PCI. Moreover, conventional wire crossing techniques fail in 40-63% of chronic total occlusion cases, and advanced re-entry and retrograde chronic total occlusion PCI techniques are complicated, time consuming, and have distinct failure mechanisms. There is an immediate need for novel technologies to simplify and improve chronic total occlusion PCI to broaden patient access to treatment and to increase procedural success.

In wire-uncrossable chronic total occlusions, advanced operators use a Controlled Antegrade and Retrograde Tracking (CART) technique to open the artery by tracking “subintimal” in the artery wall to connect the upstream and downstream artery lumen. Typical CART involves delivery of retrograde and antegrade wires into the subintimal space of the wall of the artery containing the chronic total occlusion, followed by inflation of a subintimal balloon to disrupt tissues planes to allow a subintimal wire to re-enter into the artery true lumen. Unfortunately, wire re-entry fails in about 30% of CART cases. New innovative devices are needed to improve chronic total occlusion CART reliability, efficiency, and safety.

Chronic total occlusions are often long and characterized by organized occlusive thrombi that result from plaque ruptures with subsequent healing. Peripheral chronic total occlusions frequently have severe calcium deposition that prevents luminal chronic total occlusion crossing with angioplasty wires. Endovascular approaches to peripheral chronic total occlusion revascularization are markedly challenging and have high failure rates compared to non-chronic total occlusion PCI. Open surgical bypass is typically recommended for complex chronic total occlusion lesions.

Because chronic total occlusion PCI is technically challenging, only a small number of U.S. centers offer advanced revascularization methods for coronary and peripheral chronic total occlusion treatment. The low number of focused chronic total occlusion programs nationally leads to a scenario where chronic total occlusion patients have limited access to interventional therapy and are, thus, only managed with medical therapy, which is often ineffective. Although bypass surgery can be an option, many chronic total occlusion patients are not surgical candidates based on anatomy and/or comorbidities, or because they already had bypass and the grafts have failed. Lack of access to advanced chronic total occlusion intervention results in significant morbidity from conditions such as angina, heart failure, claudication, and chronic limb ischemia where patients suffer from increased risk of outcomes like of amputation and/or cardiovascular death. As such, there is an immediate need for novel technologies to simplify and improve chronic total occlusion PCI to broaden patient access to treatment and to increase procedural success.

### SUMMARY OF THE INVENTION

The present invention relates to a magnetically guided re-entry catheter system, configured to facilitate a ferromagnetic guidewire crossing into and out of a subintimal space of an artery, such that the guidewire can bypass a chronic total occlusion of said artery. The system can comprise a catheter, a magnet, a first guidewire, and a second guidewire. The second guidewire can comprise a ferromagnetic material. The catheter is configured to be directed along the first guidewire. The magnet is affixed to a distal portion of the catheter, such that, when in use, the magnet can be brought into relatively proximity to the chronic total occlusion. The magnet force of the magnet can draw the second guidewire to the magnet when the second guidewire is in range of the magnet. In some embodiments, the second guidewire can be energized such that the second guidewire can cut tissue while crossing into and out of the subintimal space. In further embodiments, the system can further comprise electrodes and a grounding pad, wherein the electrodes are affixed to the catheter. In embodiments, the first guidewire, second guidewire, electrodes, and grounding pad can be electrically connected or disconnected in various configurations, as suitable for a particular patient or application of the present invention.

The magnetic CART crossing system will ensure expedient and precise re-entry from the subintimal tissue space into the true lumen to enable angioplasty and stenting of the CTO stenosis. Once antegrade and retrograde subintimal wire access is established, the proposed magnetized catheter will pass through the guide catheter over the subintimal wire. An energized ferromagnetic guidewire will then advance from the opposite direction through a torqueable CTO microcatheter. Once the ferromagnetic CTO wire is in the vicinity of the magnetized catheter, a purpose-built electrosurgical generator will energize the tip of the wire to enable it to cut through the tissue plane as it moves directionally by magnetic attraction toward the magnetic catheter. This electrocautery assisted/magnetic field directed rendezvous maneuver will increase the precision, speed, reliability, and safety of retrograde CTO PCI crossing.

In an embodiment, the magnetic cautery re-entry system will include: (1) an antegrade magnetic catheter which is torqueable, push-able, contains an integrated tubular high strength magnet, and acts as the anode of the electrosurgical generator; (2) a retrograde electrified guidewire which will act as the cathode of the electrosurgical generator and is steerable, lubricious, has a high tip load, and integrates an intermediate, electrically insulating polymer cover; and (3) a retrograde microcatheter and antegrade guidewire.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B are perspective views of a magnetically guided re-entry catheter system, according to an embodiment.

FIG. 1C is a schematic of a magnetically guided re-entry catheter system, accordingly to the embodiment of FIGS. 1A-1B.

FIGS. 1D-1E are perspective views of a magnetically guided re-entry catheter system, according to an embodiment.

FIGS. 2A-2E are perspective views of a magnetically guided re-entry catheter system, according to an embodiment.

FIGS. 3A-3C are schematics of a magnetically guided re-entry catheter system bypassing a chronic total occlusion within an artery, according to an embodiment.

FIG. 4 is a schematic of an alternate magnetically guided re-entry catheter system bypassing a chronic total occlusion within an artery . . .

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

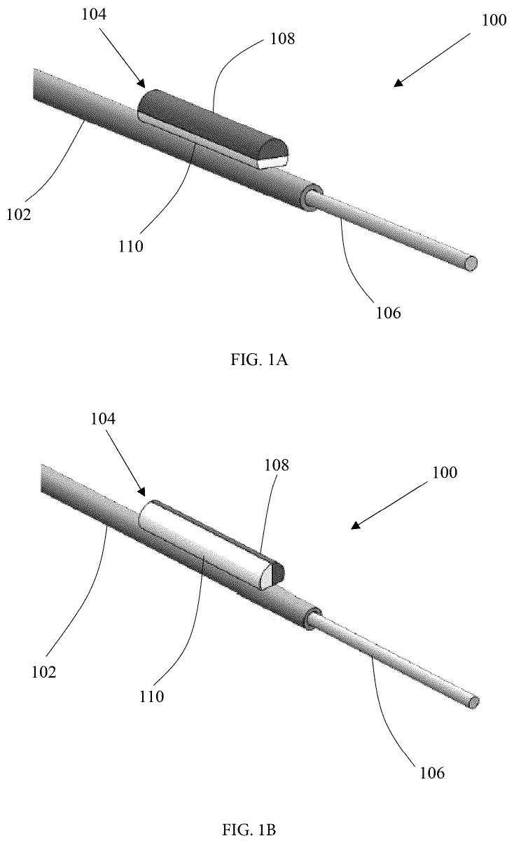

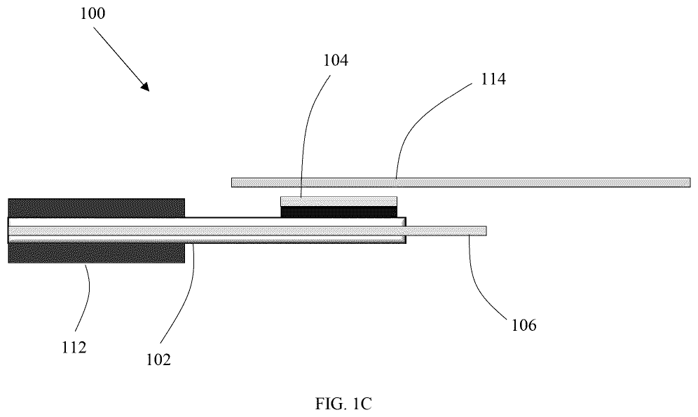

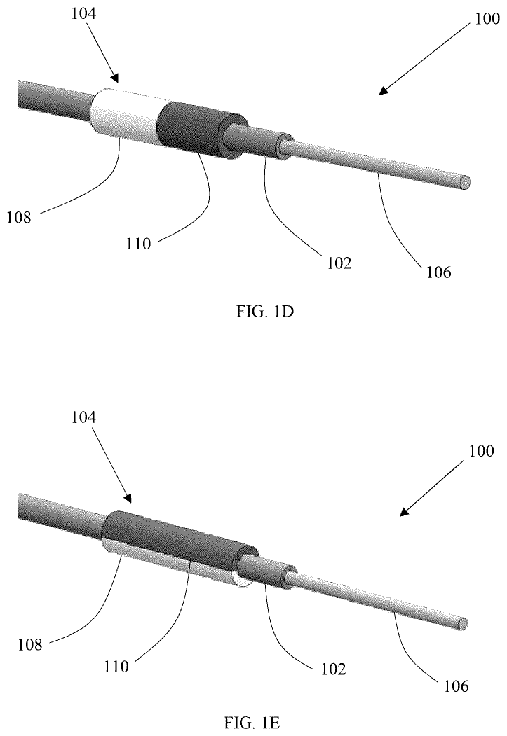

Embodiments disclosed herein can include a magnetically guided re-entry catheter system, the system comprises a ferromagnetic guidewire tip configured to direct subintimal crossing and re-entry into the artery true lumen during retrograde chronic total occlusion. Referring to FIGS. 1A-1E, an embodiment of a magnetically guided re-entry catheter system 100 is depicted. The system 100 comprises a catheter 102, a magnet 104, and a guidewire 106. The magnet 104 has a first pole 108 and a second pole 110, wherein the second pole 110 is affixed to the catheter 102. In other embodiments, the first pole 108 and the second pole 110 of the magnet 104 can be oriented perpendicular to the catheter 102, such that at least a portion of the first pole 108 and at least a portion of the second pole 110 is affixed the catheter 102. It is contemplated that the polarity of the magnet 104 can take a variety of orientations, suitable to the particular application of the disclosed device, including embodiments where the magnet 104 encompasses the catheter 102, as depicted in FIGS. 1D-1E.

Referring now to FIG. 1C, the catheter system 100 is configured to be inserted into an artery (not depicted), such as the carotid artery. The catheter 102 travels along the guidewire 106, through a guide extension system 112, such that the magnet 104 approaches one side of a chronic total occlusion (not pictured) blocking flow within said artery. On the other side of the chronic total occlusion, within the same artery, a second guidewire 114 is inserted and guided into said artery. The second guidewire 114 can comprise an energized ferromagnetic guidewire. The second guidewire 114 is configured to be inserted into a subintimal space (not pictured) of the artery. As the second guidewire 114 is positioned closer to the magnet 104, the ferromagnetic properties of the second guidewire 114 will be attracted to the magnet 104 and draw the second guidewire back out of the subintimal space, successfully bypassing the chronic total occlusion. As discussed further below, the second guidewire 114 can be configured to cut tissue within the subintimal space.

In the embodiments depicted in FIGS. 1A-1E, the guidewire 106, the second guidewire 114, and a grounding pad (not depicted) are configured to be electrodes or be disconnected. Below, Table 1 indicates the various orientations that may be suitable for these embodiments. It is contemplated that a wide variety of other configurations may be implemented as well, as suitable for the particular application or patient.

TABLE 1

| Guidewire | Second Guidewire | Grounding Pad |
|---|---|---|
| Not connected | Cathode | Anode |
| Anode | Cathode | Not connected |
| Anode | Cathode | Anode |
| Cathode | Anode | Not connected |

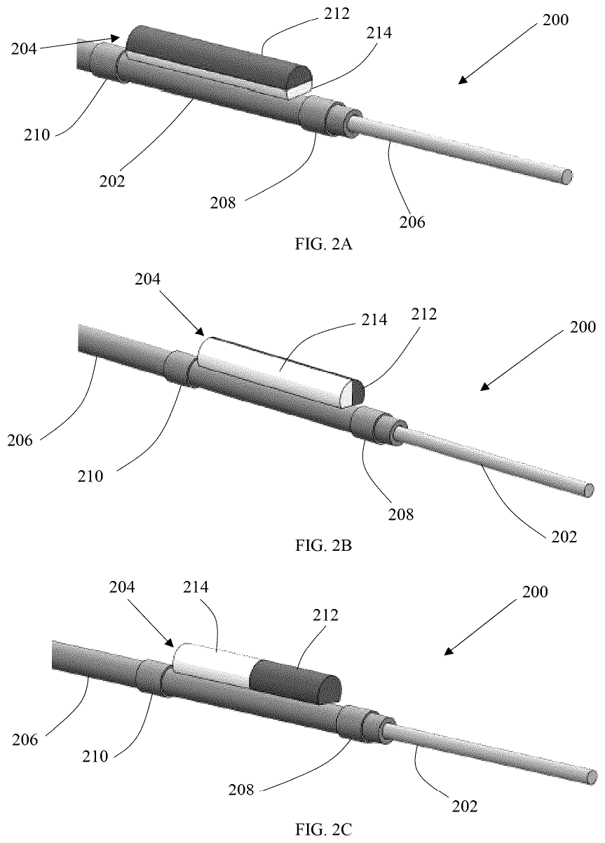

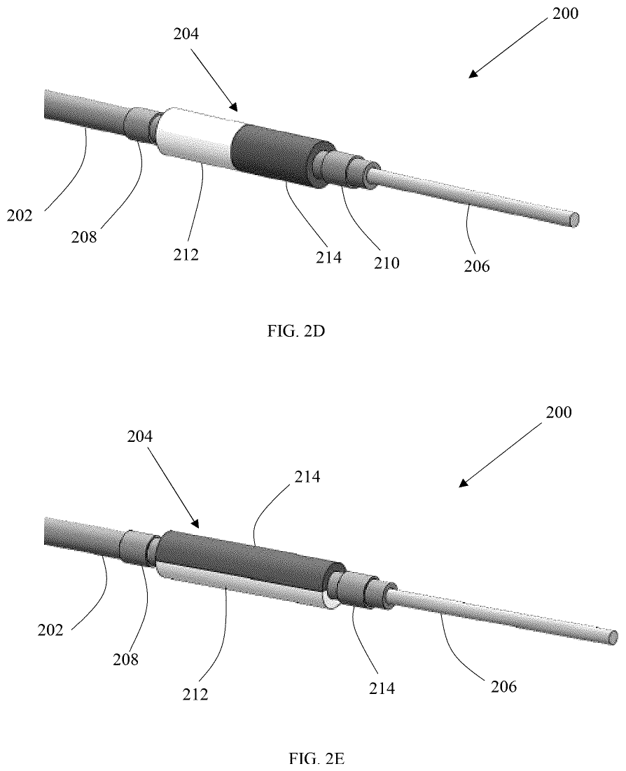

Referring generally to FIGS. 2A-2E, a magnetic controlled antegrade and retrograde tracking catheter system 200 can comprises a catheter 202, a magnet 204, a guidewire 206, a first electrode 208, and a second electrode 210. The magnet 204 comprises a first pole 212 and a second pole 214, wherein the magnet 204 is affixed generally to the catheter 202. It is contemplated that the magnet 204 can comprise a variety of polarity orientations, such as where the magnet 104 can be affixed to a discrete portion of the catheter 202 (as depicted in FIGS. 2A-2C) or where the magnet 204 encompasses the catheter 202 (as depicted in FIGS. 2D-2E). While FIGS. 2A-2E depict an embodiment with two electrodes, it is contemplated that any number of electrode may be positioned proximal or distal to the magnet 204, in accordance with the technical needs of the application. The electrodes may take any shape and be electrically connected via discrete wires or via the guidewire 206.

Similar to the embodiments depicted in FIGS. 1A-1E, the catheter system 200 is configured to be inserted into an artery (not depicted). The catheter 202 can be directed along the guidewire 206, such that the catheter 202 moves through the artery and approaches a chronic total occlusion (not depicted). In such an embodiment, as described supra, a second guidewire is inserted into the artery, such that the second guidewire approaches the chronic total occlusion from the opposite side as the catheter 202. The second guidewire can comprise a ferromagnetic material. The second guidewire is directed into the subintimal space (not depicted) of the artery, such that the second guidewire can bypass the chronic total occlusion. Once the second guidewire is in range of the magnet 204, the second guidewire can be energized such that the second guidewire can cut through the tissue of the subintimal space and be drawn back into the artery by the magnetic force of the magnet 204. In such an embodiment, the guidewire, the second guidewire, a grounding pad (not depicted), and the electrodes can be electrically connected or disconnected, depending on the requirements of a particular application of the device. Table 2 below lists six example contemplated, but additional configurations are contemplated.

TABLE 2

| Guidewire | Second Guidewire | Electrodes | Grounding Pad |
|---|---|---|---|
| Anode | Cathode | Anode | Not connected |
| Anode | Cathode | Anode | Anode |
| Not connected | Cathode | Anode | Not connected |
| Not connected | Cathode | Anode | Anode |
| Cathode | Anode | Cathode | Not connected |
| Not connected | Anode | Cathode | Not connected |

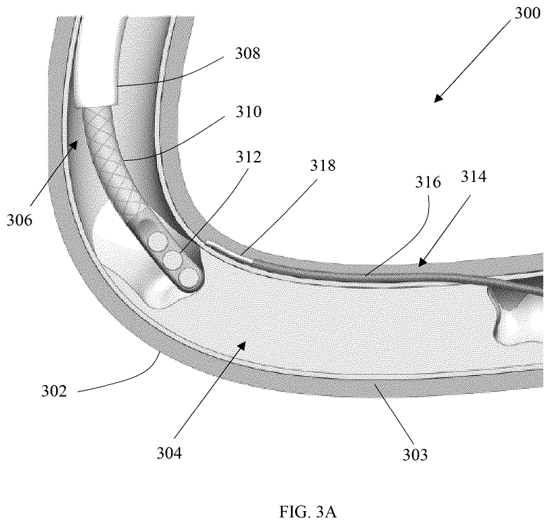

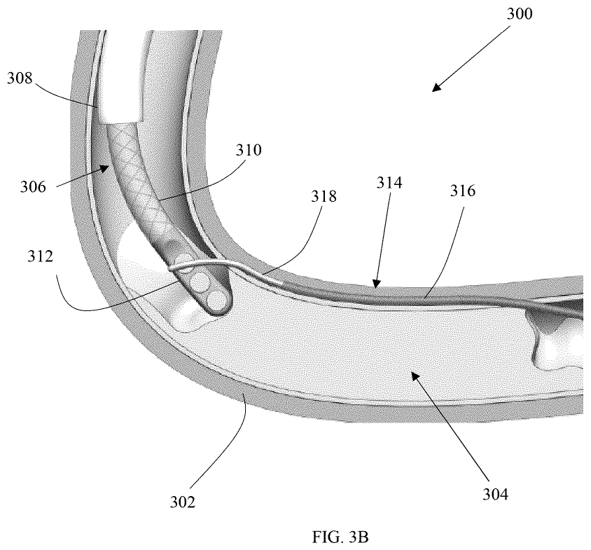

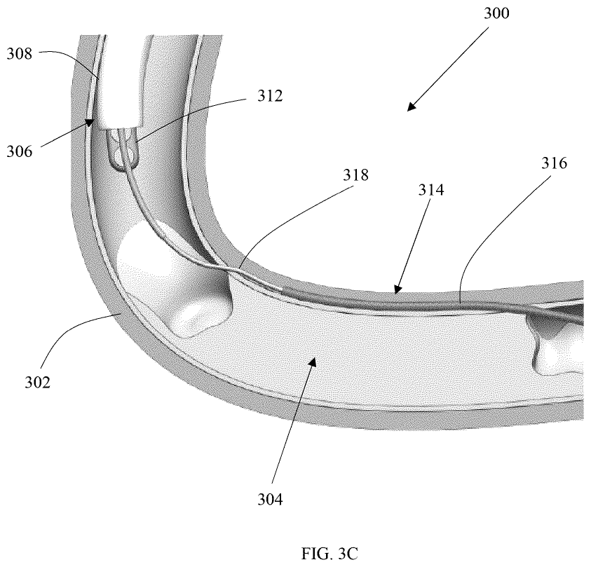

FIGS. 3A-3C illustratively show a model of the present invention in use. In the example, an artery 302 is depicted, with a plaque blockage 304. The plaque blockage 304 is total, completely blocking the flow of blood through the artery 302. The artery 302 further comprises a subintimal crossing 303. A magnetic antegrade and retrograde catheter system 300 has been inserted into the artery 302, such that a magnetic portion 306 approaches the plaque blockage 304 from one direction and a guidewire portion 314 approaches the plaque blockage 304 from the other direction.

The magnetic portion 306 can comprise a magnetized tip 312, a catheter 310, and a guide catheter 308. The catheter 310 of the magnetic portion can be a 3 to 6 French (F) magnetic tip nylon catheter. The antegrade magnetic catheter in one embodiment may include a diametric, tube-shaped magnet of 5 French OD and a length selected for high side facing magnetic force and axial symmetry.

In some embodiments, the catheter may be extruded nylon wrapped in a women stainless steel braiding and coated in a hydrophilic PTFE coating. Such an embodiment provides optimal flexibility, torqueability, and lubricity, while remaining resisting to kinking in the catheter 310. In choosing an appropriate magnet for the magnetized tip 312, it is important to consider the strength of the magnet, to ensure that it is strong enough to attract the guidewire portion 314, but low enough to not attract stents or other implantable devices. For example, the magnetized tip 312 can comprise a nylon capsule configured such that three neodymium magnets can be affixed to the magnetized tip 312.

In an embodiment, a combination of stainless-steel braids and coils may be wound around the mandrel to give the catheter torque-ability, push-ability, and kink resistance. A platinum iridium wire may be placed around the distal catheter segment in a shape that indicates the magnet polarity under angiography. An insulative, lubricious, and flexible polymer outer layer such as Nylon may be applied over the braids and coils using a reflow process. On the distal segment of the catheter, the polymer layer may be removed, and a custom neodymium magnet may be bonded to the stainless-steel layer in alignment with the polarity indicators. A soft, atraumatic polymer tip will be reflowed onto the distal catheter end. Finally, on the proximal catheter end, a custom hub with an electrical connector may be attached to facilitate conductivity through the stainless-steel braid and coil layer to the magnet on the distal catheter end.

The guidewire portion 314 can comprise a ferromagnetic guidewire 318 and a microcatheter 316, wherein the ferromagnetic guide 318 resides in an internal passage of the microcatheter 316, such that the ferromagnetic guidewire can emerge from an open end of the microcatheter 316.

In an embodiment, the guidewire may comprise a stainless steel or nitinol wire stock drawn to the target core diameter and heat treated to maximize steerability. The distal segment of the wire may be tapered to enhance flexibility using a precision centerless grinding process. To achieve the high tip load and enhance the attraction to the magnetic catheter, a core-to-tip anatomy will be employed, and a radiopaque, magnetic spring coil may be wound around the distal end of the core wire.

The microcatheter 316 can comprise a material that is electrically insulated and torqueable. Like the catheter 310, the microcatheter must similarly comprise materials designed to optimize the flexibility, torqueability, and lubricity of the exterior of the microcatheter 318, while resisting kinking. However, due to the small size of the microcatheter 318, it is contemplated that extrusion may not be a suitable production method. In embodiments where extrusion is not a suitable production method, the microcatheter 318 will be manufactured using dip coating instead of extrusion. A thin layer of PTFE can be coated and cured on a mandrel the desired length of the guidewire portion 314. Then, a stainless-steel braining will be wound around the mandrel to give the microcatheter 318 strength, pushablility, and torqueability. Finally, the PTFE braid will be dipped through polyimide and cured, to create the final microcatheter 318.

In practice, as demonstrated in FIGS. 3A-3C, the magnet portion 306 in inserted into the true lumen space of the artery 302, such that the magnetic portion is situated on one side of the blockage 304. The guidewire portion 314 is inserted first into the true lumen of artery 302, to approach the area of the artery 302 where the plaque blockage 304 resides, before exiting the lumen of the artery 302 into the subintimal space 303. The ferromagnetic guidewire 318 is maneuvered through the subintimal space 303, until is reaches the range of the magnetized tip 312.

Once the ferromagnetic guidewire 318 is within range of the magnetized tip 312, the ferromagnetic guidewire 318 can be energized by an electrosurgical generator (not depicted). The electrosurgical generator can be operated in the range of 30-120 watts in the 300-500 kHz frequency range. Energizing the tip enables the ferromagnetic guide wire 318 to cut through the tissue plane of the subintimal space 303, such that it reenters the lumen of the artery 302 on the same side of the plaque blockage 304 as the magnetic portion 306. It is envisioned that electrocautery type may include monopolar cut, monopolar coagulate, bipolar coagulate and multiple anode/cathode configurations. In an embodiment, clean cut were produced using a monopolar cut, retrograde wire as the cathode.

The ferromagnetic guidewire 318 is moved directionally by the magnetic attraction towards the magnetic portion 306 of the system 300. In embodiments, the ferromagnetic guidewire 318 can be energized using 50 W of power for short, one second bursts. It is contemplated that other levels of energizing may be suitable, including 20 W which strikes a balance between cutting speed and safety. Cutting may depend on the patient and the circumstances particularly to that patient.

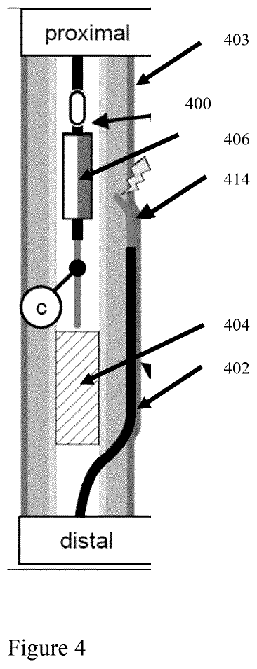

FIG. 4 illustrates an alternate model of the present invention in use. In the example, an artery 402 is depicted, with a plaque blockage 404. The plaque blockage 404 is total, completely blocking the flow of blood through the artery 402. Artery 402 further comprises a subintimal crossing 403. A magnetic antegrade and retrograde catheter system 400 has been inserted into the artery 402, such that a magnetic portion 406 approaches the plaque blockage 404 from one direction and an electrified cathode guidewire portion (C) 414 approaches the plaque blockage 404 from the other direction.

In an embodiment, the self-contained, battery-operated catheter system will incorporate a microcontroller and software to power the catheter system, monitor the procedure, and deliver electrical cauterization pulses during use. The energy delivery system may be optimized to provide a minimum of 20 ten-second bursts of 20 W-cut electrocauterization pulses, the number and type of pulses needed to perform a full procedure plus a safety factor.

To minimize surgical risk of perforations, the system will include safeguards. Because the antegrade magnetic catheter acts as the anode, the path of least electrical resistance, and therefore most efficient cutting, is the tissue plane directly between the magnetic catheter and the retrograde electrified guidewire, meaning it is easiest to cut in the intended direction. The battery-powered electrosurgical generator will not have a path to earth ground and thus will minimize hazardous current pathways for the patient and the surgical staff. The energy needed for the entire procedure will be stored in disposable primary batteries, which will use a voltage boost system to transfer electrical energy to a bank of capacitors that can deliver high-voltage current-limited pulses during the treatment cycles. The capacitor bank cannot be charged instantly, so more cauterization pulses become available as elapsed time increases. We expect that users will discharge the cauterization pulses at a rate equal to or less than 10 pulses in the first 5 minutes. A simple user interface on the device displays the number of cauterization pulses that are currently available.

Both the voltage and current will be monitored by the control system during treatment, so the impedance of the tissue plane can be computed. If the impedance is relatively high and continues to increase during a treatment cycle, the electrified guidewire may be moving the wrong direction, and pulses will be interrupted before significant damage is caused to unintended tissues. If the impedance is in the desired range and decreases during the treatment, the electrified guidewire is making progress. When the rendezvous occurs, the impedance drops sharply, the pulsing will be stopped, and a successful rendezvous indicated to the operator.

In an embodiment, the wire shape for the rendezvous maneuver may be with a primary, sharp, 45° bend ˜1 mm from the tip and secondary gentle bend in the same direction. While varied length of exposed retrograde wire may be used, cleanest, fastest cuts were found with <3 mm of wire exposed. Successful rendezvous maneuvers were consistent at <5 mm distance.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A magnetically guided re-entry catheter system, configured to bypass a chronic total occlusion within an artery, the system comprising:
a catheter;
at least one magnet;
a first guidewire configured to direct the catheter through the artery;
a second guidewire comprising a ferromagnetic tip, wherein the second guidewire is configured to be magnetically attracted to the at least one magnet.

2. The magnetically guided re-entry catheter system of claim 1, the system further comprising at least one electrode, wherein the at least one electrode can be electrically connected or disconnected.

3. The magnetically guided re-entry catheter system of claim 1, the system further comprising a grounding pad, wherein the grounding pad can be electrically connected or disconnected.

4. The magnetically guided re-entry catheter system of claim 1, the system further comprising an electrosurgical generator configured to energize the second guidewire, such that the second guidewire can cut through tissue upon crossing into and out of a subintimal space of the artery.

5. The magnetically guided re-entry catheter system of claim 1, wherein the at least one magnet is affixed to a distal portion of the catheter.

6. The magnetically guided re-entry catheter system of claim 1, the system further comprising a capsule affixed to the distal end of the catheter, wherein the capsule is configured to contain or receive the at least one magnet.

7. The magnetically guided re-entry catheter system of claim 1, the system further comprising a microcatheter encapsulating the second guidewire.

8. The magnetically guided re-entry catheter system of claim 1, wherein the first guidewire and the second guidewire can be electrically connected or disconnected.

9. The magnetically guided re-entry catheter system of claim 1, wherein the second guidewire is energized using a discrete wattage of power for repeating periods of time.

10. The magnetically guided re-entry catheter system of claim 1, the system further comprising a guide extension system configured to assist in directing the catheter through the artery.

11. A method for bypassing a chronic total occlusion within an artery, the method comprising:
inserting a catheter into the artery, wherein at least one magnet is affixed to a distal portion of the catheter and wherein a first guidewire extends through an internal passage of the guidewire;
directing the catheter through the artery, such that the magnet is positioned on one side of the chronic total occlusion;
inserting a second guidewire into the artery from the opposite direction that the catheter was inserted, wherein the second guidewire is configured to be magnetically attracted to the magnet;
directing the second guidewire to a second side of the chronic total occlusion;
crossing the second guidewire into a subintimal space of the artery, such that the second guidewire can bypass the chronic total occlusion;
directing the second guidewire through the subintimal space of the artery until the second guidewire is within the magnetic field of the magnet;
crossing the second guidewire out of the subintimal space of the artery, wherein the second guidewire is directionally moved by the magnet force between the second guidewire and the magnet.

12. The method for bypassing a chronic total occlusion within an artery of claim 11, wherein the catheter further comprises at least one electrode and wherein the electrode is electrically connected or disconnected.

13. The method for bypassing a chronic total occlusion within an artery of claim 11, the method further comprising energizing the second guidewire with an electrosurgical generator.

14. The method for bypassing a chronic total occlusion within an artery of claim 11, the method further comprising cutting the tissue of the subintimal space with the second guidewire.

15. The method for bypassing a chronic total occlusion within an artery of claim 11, the method further comprising energizing the second guidewire with a discrete wattage of power for repeating periods of time (i.e. every one second).

16. The method for bypassing a chronic total occlusion within an artery of claim 11, the method further comprising electrically connecting or disconnecting the first guidewire and the second guidewire, according to the desired electrical configuration.

17. The method for bypassing a chronic total occlusion within an artery of claim 11, the method further comprising electrically connecting or disconnecting a grounding pad, according to the desired electrical configuration.

18. The method for bypassing a chronic total occlusion within an artery of claim 11, wherein the second guidewire is encapsulated by a microcatheter.

19. The method for bypassing a chronic total occlusion within an artery of claim 11, wherein the catheter is coupled to a guide extension system configured to assist with directing the catheter through the artery.

20. The method for bypassing a chronic total occlusion within an artery of claim 11, wherein the catheter a polymeric layer, a steel braiding layer, and thermoset layer.
